(19) United States
(12) Patent Application Publication
(10) Pub. No.: US 2026/0305942 A1
(43) Pub. Date: Oct. 8, 2026

(54) MEDICAL SYSTEM CONFIGURED TO USE SOURCE SEPARATION TO IDENTIFY PATIENT PARAMETERS FROM SIGNALS RECEIVED FROM MULTIPLE MEDICAL DEVICES

(71) Applicant: Medtronic, Inc., Minneapolis, MN (US)

(72) Inventors: Richard J. O'Brien, Hugo, MN (US); Saul E. Greenhut, Denver, CO (US); Yong K. Cho, Excelsior, MN (US); Can Cinbis, Salt Lake City, UT (US); Sean R. Landman, Minneapolis, MN (US); Shantanu Sarkar, Roseville, MN (US); Mirko de Melis, Maastricht (NL); Tyler W. Stigen, Brooklyn Park, MN (US); Randal C. Schulhauser, Phoenix, AZ (US)

(21) Appl. No.: 19/479,000

(22) PCT Filed: Apr. 1, 2024
(86) PCT No.: PCT/IB2024/053153

§ 371 Date: Oct. 27, 2025

Related U.S. Application Data
(60) Provisional application No. 63/498,965, filed on Apr. 28, 2023.

(51) Int. Cl. A61B 5/00; A61B 5/318

(52) U.S. Cl. CPC A61B 5/0011; A61B 5/318; A61B 5/6814; A61B 2503/02

(57) ABSTRACT

An example system includes a first implantable medical device (IMD), a second medical device, and processing circuitry. The first IMD configured to receive a first sensed signal from one or more first sensors. The second medical device configured to receive a second sensed signal from one or more second sensors. The processing circuitry configured to receive the first sensed signal and the second sensed signal; synchronize timing of the first sensed signal and the second sensed signal; perform source separation signal processing on the first sensed signal and second sensed signal to separate a first source signal and a second source signal from each other; and determine a health condition status of a patient based at least in part on one or more of the separated first source signal or the separated second source signal.

### FIELD

This application claims the benefit of U.S. Provisional Patent Application Ser. No. 63/498,965, filed Apr. 28, 2023, the entire content of which is incorporated herein by reference.

### FIELD

This disclosure generally relates to systems including medical devices and, more particularly, to monitoring of patient health using such systems.

### BACKGROUND

A variety of devices are configured to monitor physiological signals of a patient. Such devices include implantable or wearable medical devices, as well as a variety of wearable health or fitness tracking devices. The physiological signals sensed by such devices include as examples, electrocardiogram (ECG) signals, electroencephalograph (EEG) signals, heart sounds, respiration signals, perfusion signals, activity and/or posture signals, pressure signals, blood oxygen saturation signals, body composition, fluid impedance signals, and blood glucose or other blood constituent signals. In general, using these signals, such devices facilitate monitoring and evaluating patient health over a number of months or years, outside of a clinic setting.

### SUMMARY

In general, the disclosure describes techniques involving a system having a plurality of sensors to monitor/record signals of a same type from different locations to determine physiologic parameters of a patient. The system obtains a first sensed signal from a first sensor, the first sensed signal including a first source signal and a second source signal, and obtains a second sensed signal from a second sensor, the second sensed signal including a first source signal and the second source signal. The system is configured to perform source separation signal processing to separate the first source signal and the second source signal from each other, and then determine a first physiological parameter based on the first source signal and/or determine a second physiological parameter based on the second source signal.

Unlike conventional detection systems, the techniques and systems of this disclosure may use source separation signal processing to separate each of the source signal(s) from each other to determine a health condition status, which improves the accuracy and usefulness of the determination of the physiological parameters.

Additionally, the techniques and systems of this disclosure may be implemented using one or more implantable medical devices (IMDs) that can continuously and/or periodically sense signals, such as ECG signals, without human intervention while subcutaneously implanted in a patient over months or years, allowing the system to perform thousands to millions of operations per second to synchronize the signals from the devices and perform source separation signal processing to separate the source signal(s) from each other. Using techniques of this disclosure in a system or device may be advantageous when a physician or other health care professional (HCP) cannot be continuously present with the patient over weeks or months to evaluate and separate source signals from signals from multiple devices from each other and determine a health condition status based on the separated source signals, such ECG or EEG data, and/or where performing millions of operations on weeks or months of signal data, such as ECG or EEG data, could not practically be performed in the mind of a physician or other HCP with techniques of this disclosure that synchronize the obtained signals, perform source separation signal processing to separate the source signal(s) from each other, and determine a health condition status based on the separated source signal(s).

Synchronizing the obtained sensed signals from different medical devices and performing source separation signal processing to separate the source signal(s) from each other implementing techniques of this disclosure may provide one or more technical and clinical advantages. For example, when a determined physiological parameter is based on a source signal that has smaller amplitude than the other source signal(s), the techniques described herein may help obtain the source signal with a smaller amplitude, that may appear to be a noise signal in conventional detection systems, and enable the system to determine a health condition status based on the separated source signal having a smaller amplitude than the other source signal(s).

In one example, the disclosure describes a system comprising a first implantable medical device (IMD) comprising one or more first sensors, the first IMD configured to receive a first sensed signal from the one or more first sensors, the first sensed signal including a first source signal and a second source signal; a second medical device comprising one or more second sensors, the second medical device configured to receive a second sensed signal from the one or more second sensors, the second sensed signal including the first source signal and the second source signal; and processing circuitry configured to: receive the first sensed signal and the second sensed signal; synchronize timing of the first sensed signal and the second sensed signal; perform source separation signal processing on the first sensed signal and second sensed signal to separate the first source signal and the second source signal from each other; and determine a health condition status of a patient based at least in part on one or more of the separated first source signal or the separated second source signal.

In another example, this disclosure describes a method for operating processing circuitry of a computing device, the method comprising: receiving, by the processing circuitry and via a first implantable medical device (IMD) implanted in a patient, a first sensed signal including a first source signal and a second sensed signal; receiving, by the processing circuitry and via a second medical device coupled to the patient, a second sensed signal including the first source signal and the second source signal; synchronizing, by the processing circuitry, timing of the first sensed signal and the second sensed signal; performing, by the processing circuitry, source separation signal processing on the first sensed signal and second sensed signal to separate the first source signal and the second source signal from each other; and determining, by the processing circuitry, a health condition status of the patient based at least in part on one or more of the separated first source signal or the separated second source signal.

In another example, the disclosure describes a computing device comprising: a memory; and processing circuitry coupled to the memory; the processing circuitry being configured to: receive, via a first implantable medical device (IMD), a first sensed signal including a first source signal and a second source signal; receive, via a second medical device, a second sensed signal including the first source signal and the second source signal; synchronize timing of the first sensed signal and the second sensed signal; perform source separation signal processing on the first sensed signal and second sensed signal to separate the first source signal and the second source signal from each other; and determine the health condition status of the patient based at least in part on one or more of the separated first source signal or the separated second source signal.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system configured to determine a health condition status of a patient in accordance with one or more techniques of this disclosure.

FIG. 2 is a block diagram illustrating an example configuration of an implantable medical device that operates in accordance with one or more techniques of the present disclosure.

FIG. 3 is block diagram illustrating an example configuration of a computing device that operates in accordance with one or more techniques of the present disclosure.

FIG. 4A is a conceptual side-view diagram illustrating an example configuration of an IMD of FIG. 2.

FIG. 4B is a conceptual side-view diagram illustrating an example configuration of an IMD of FIG. 2.

FIG. 5 is a block diagram illustrating an example system that includes implantable medical device(s) and/or computing device(s) used to determine a health condition status of a patient.

FIG. 6 is a flow diagram illustrating an example operation to determine a health condition status of a patient.

FIG. 7 is a diagram illustrating an example operation with a plurality of implantable medical devices positioned in a patient with a fetus.

Like reference characters refer to like elements throughout the figures and description.

### DETAILED DESCRIPTION

Physiologic signals are important to assess a variety of events related to a patient, such as patient's health, a therapy's effectiveness, and/or disease progression. Because of multiple physiologic signal sources, noise sources, or low signal level it is often difficult extract physiologic parameters of interest from a single signal sensed from a single sensor which may be desired to diagnose or track certain illnesses, conditions, or overall health. This problem spans many sensors (e.g., ECG, EEG, heart sounds, etc.) and many clinical applications.

The body is a complex system and there are a limited number of ways in which physiologic signals may safely be detected. Quality of a signal from a physiological sensor may be related to a location of the physiologic sensor, but there may be a limited number of locations on the body for detecting signals due to limitations on where implantable medical devices may be implanted and/or where external devices may be located in relation to a source of a signal a respective device may be configured to detect.

Clinical measurements have generally been taken in a controlled environment while the patient is still with sensors carefully placed to reduce possible sources of signal error. Even with careful sensor placement for optimum signal detection, a strong signal may overpower the effects of a weaker signal. For example, EEG signal processing may filter out ECG signals or signals from muscle movement, such as blinking, chewing, or head turning.

However, most measurements taken by ambulatory sensors, whether wearable or implantable, will not be taken under the relatively ideal conditions of a clinical setting as a purpose of these new sensors is to collect physiologic signals outside of the clinical setting. For long term use, a location of a wearable device may be limited by a variety of factors, such as comfort considerations. For an implantable medical device, factors such as size of the implantable medical device and other considerations, such as communications or battery recharging, may limit the implantable medical device and sensor location.

If sensors cannot be placed in such a way that only the signal(s) of interest is collected the noise signals must be isolated and removed by filtering or signal processing. In accordance with techniques of this disclosure, a system is configured to obtain a first sensed signal from a first sensor, the first sensed signal including a first source signal and a second source signal, and obtain a second sensed signal from a second sensor, the second sensed signal including the first source signal and the second source signal. The system is configured to perform signal processing, such as source separation signal processing, to separate the first source signal and the second source signal from each other, and then determine a first physiological parameters based on the first source signal and determine a second physiological parameter based on the second source signal.

In some examples, a system may perform source separation signal processing using signals collected from multiple sensors from a plurality of devices, such as two or more implantable medical devices or one implantable medical device and a wearable device, and isolate each individual signal source from the other. In some examples, the system described herein may separate the two or more source signals when the signals cannot be separated using standard filtering techniques which rely on differences in signal frequency content. In some examples, a system, as described herein, separating source signals, for a sensor in each device, from each other using source separation signal processing may improve accuracy of a determination of a health condition status of a patient and may also enable implantable medical devices and/or wearable devices to be positioned at more locations while still being able to determine a health condition status accurately, which may improve the reliability and usefulness of the system.

A variety of types of implantable and external devices may be configured to detect a status of health conditions based on sensed physiological parameters. External devices that may be used to non-invasively sense and monitor physiological parameters include wearable devices with electrodes configured to contact the skin of the patient, such as patches, watches, rings, necklaces, hearing aids, clothing, car seats, or bed linens. Such external devices may facilitate relatively longer-term monitoring of patient health during normal daily activities.

Implantable medical devices (IMDs) also sense and monitor physiological parameters. In some examples, IMDs may not provide therapy, such as implantable patient monitors. One example of such an IMD is the Reveal LINQ™ or LINQ II™ Insertable Cardiac Monitor (ICM), available from Medtronic, Inc., which may be inserted subcutaneously. Some IMDs may extract features from EEG signals indicative of brain activity or cardiac activity. Some IMDs may provide therapy and may include pacemakers and implantable cardioverter-defibrillators, which may be coupled to intravascular or extravascular leads, as well as pacemakers with housings configured for implantation within the heart, which may be leadless. One example of such an IMD is the Micra™ leadless pacemaker, available from Medtronic, Inc. Such IMDs may facilitate relatively longer-term monitoring of patients during normal daily activities, and may periodically transmit collected data to a remote patient monitoring system, such as the Medtronic Carelink™ Network.

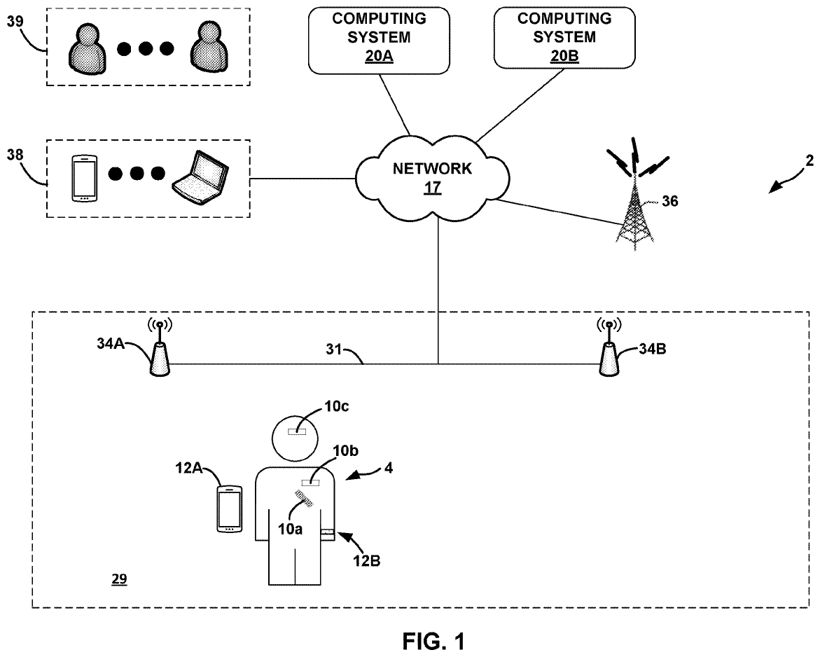

FIG. 1 is a block diagram illustrating an example system 2 configured to detect statuses of health conditions of patient 4, and to respond to such detection, in accordance with one or more techniques of this disclosure. Patient 4 ordinarily, but not necessarily, will be a human. For example, patient 4 may be an animal needing ongoing monitoring for health conditions. As used herein, the terms “detect,” “detection,” and the like may refer to detection of a status of a health condition presently (at the time or the period of time the data is collected) being experienced by patient 4, as well as detection based on the data that the condition of patient 4 is such that they have a suprathreshold likelihood of experiencing the health condition within a particular timeframe, e.g., prediction of the health condition and/or prediction in a change of the health condition. The example techniques may be used with two or more patient sensing devices, e.g., IMDs 10a, 10b, and/or 10c (collectively, “IMDs 10”), which may be in wireless communication with each other and/or may be in wireless communication with one or more patient computing devices, e.g., patient computing devices 12A and 12B (collectively, “patient computing devices 12”). In some examples, the two or more patient sensing devices 10 may be implanted within patient 4. In some examples, patient sensing devices may be external to (e.g., worn by) patient 4. For example, system 2 with two or more of IMDs 10a, 10b, 10c, or other sensing devices, such as wearable computing device 12B, may capture different values of a common patient parameter with different resolution/accuracy based on their respective locations. Although not illustrated in FIG. 1, IMDs 10 may include electrodes and other sensors to sense physiological signals of patient 4, and may collect and store sensed physiological data based on the signals and detect a health condition status based on the data. In some examples, the example techniques may be used with two or more patient sensing devices, such as at least one IMD 10a and a wearable computing device 12B, which may be in wireless communication with each other and/or may be in wireless communication with one or more patient computing devices 12A.

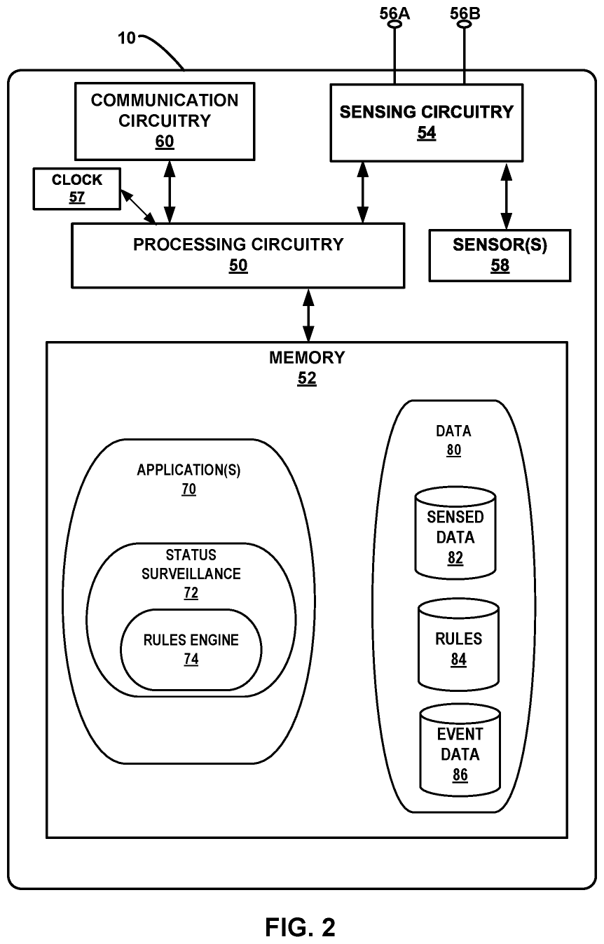

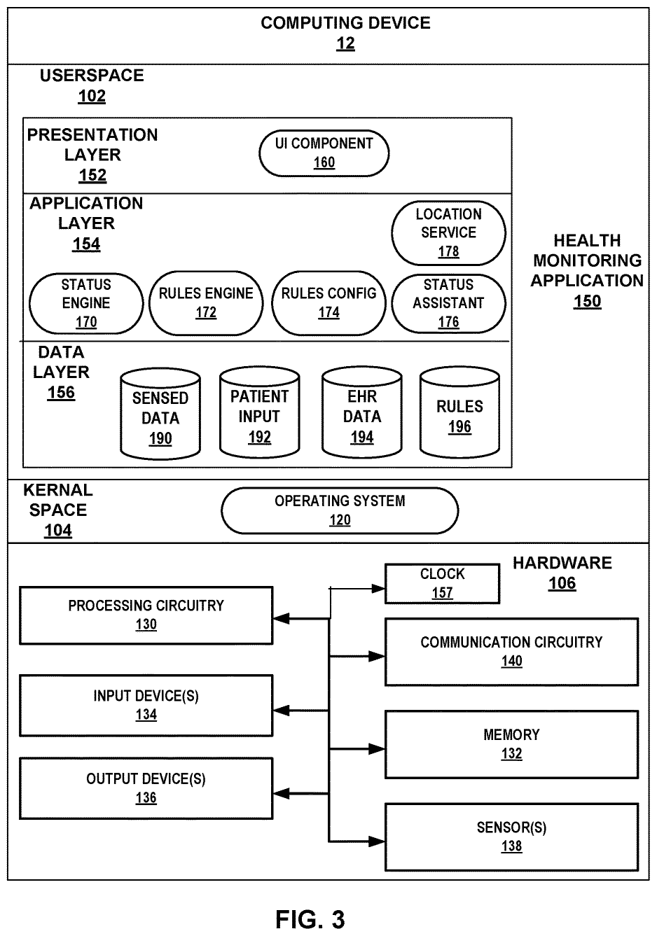

In some examples, system 2 may include a first IMD and a second medical device. In some examples, system 2 may include more than two medical devices. For simplicity, system 2 may be referenced as having two medical devices and two respective signals in the examples discussed below, but system 2 may include three or more medical devices with each receiving a respective signal and separating a source signal from the respective received signal in accordance with techniques described herein. The first IMD, such as IMD 10a, may include one or more first sensors, such as electrode 56A, 56B, and/or sensor(s) 58, as shown in FIG. 2. In some examples, a second medical device, such as IMD 10b and/or computing device 12b, may include one or more respective second sensors, such as electrode 56A, 56B, and/or sensor(s) 58 included in IMD 10, as shown in FIG. 2, or sensor(s) 138 included in computing device 12B, as shown in FIG. 3. The first IMD may be configured to receive a first sensed signal corresponding to a first parameter from the one or more first sensors. The first sensed signal includes a first source signal and a second source signal. The second medical device may be configured to receive a second sensed signal corresponding to a second parameter from the one or more second sensors. The second sensed signal includes the first source signal and the second source signal. In some examples, the first sensed signal(s) and/or the second sensed signal(s) include a sensed subcutaneous impedance.

In some examples, first IMD may be configured to be positioned in a first location of a body of the patient and the second medical device may be configured to be positioned in a second location, the first location being different than the second location. In some examples, the first IMD may be configured to be inserted subcutaneously. For example, the first location may be proximal to a heart of the patient or a head of the patient, and the second location may be proximal to a womb of the patient for a patient that is pregnant, a head of the patient (e.g., if the first location is proximal to the heart of the patient), or external to the patient.

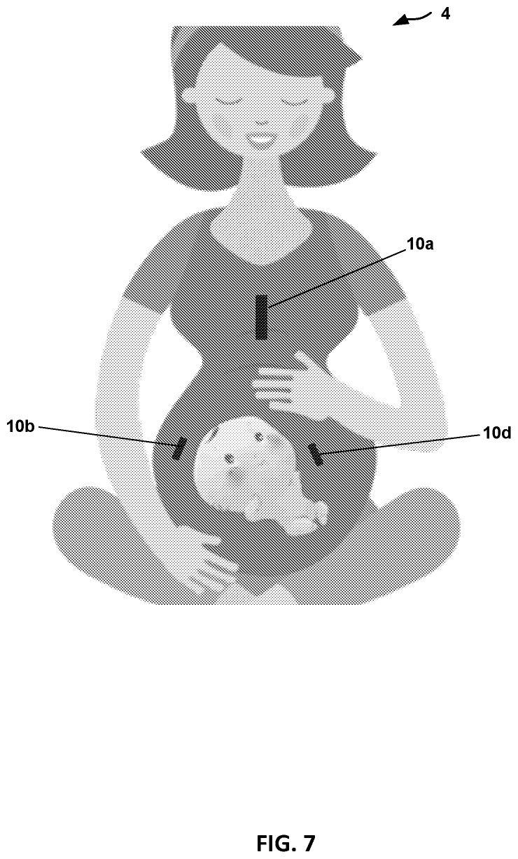

In some examples, a first IMD and/or second medical device may be positioned to be near where a desired signal is emanating from. As shown as an example in FIG. 7, if the desired signal of the first IMD 10a is an ECG of the patient and a desired signal of a second medical device 10b and/or 10d is an ECG from a fetus in the patient, a first IMD 10a may be positioned near the heart of the patient, while the second medical device 10b and/or 10d may be positioned near the fetus in the patient.

As shown in FIGS. 2, 3, and 5, processing circuitry 50, 130, 23 is located in respective IMD 10a, IMD 10b, patient computing devices 12, such as wearable computing device 12B, and computing systems 20. For simplicity, processing circuitry 50 will be referenced in the examples discussed below, but any one or more of processing circuitries 50, 130, and 23 may be used as the processing circuitry.

In some examples, processing circuitry 50 may be configured to receive the first sensed signal including a first source signal and a second source signal and the second sensed signal including the first source signal and the second source signal, perform source separation signal processing on the first sensed signal and second sensed signal to separate the first source signal and the second source signal from each other.

In some examples, the source separation signal processing may be blind source separation (BSS). Processing circuitry 50 may be configured to use principal components, wavelets, independent component analysis, and/or principal component analysis.

Independent component analysis and principal component analysis are types of BSS to decompose the signals into distinct components. As an example, filters and amplifiers may be configured to filter the wavelet-transformed signals to remove frequencies outside of desired frequency ranges before amplifying the filtered signals. Using a classification technique, noise and other artifacts can be zeroed out or ignored. Processing circuitry 50 may be configured to perform an inverse wavelet transform to create clean, reconstructed separated first source signal and separated second source signal.

An example of BSS performed by processing circuitry 50 may use multiple signals, such as the first sensed signal and the second sensed signal, with a non-stationary or maximum overlap wavelet transform or empirical mode decomposition. In this example, processing circuitry 50 may apply independent component analysis to each transformed signal. Processing circuitry 50 can classify the components of the transformed signal as a common noise signal, a first source signal, or a second source signal and separated from each other, with noise components being discarded. First source signal and second source signal components can be separated and inverse transformed generating clear separated first source signal and separated second source signal, respectively. For example, when the first source signal is an ECG signal of a patient and the second source signal in an ECG signal of a fetus in the patient, an ECG signal of the fetus may have a much lower amplitude than an ECG signal of the fetus and the second medical device directed to obtain an ECG signal of the fetus may also obtain an ECG signal from the patient. Thus, as an example, processing circuitry 50 may apply BSS to generate a clear ECG signal of the patient and a clear ECG signal of the fetus in the patient. Additional example details of BSS algorithms are described in “Issues and Research on Foetal Electrocardiogram Signal Elicitation” by Jagannath et al., published in in Biomedical Signal Processing and Control in 2013, the entire contents of which are incorporated herein by reference.

Additionally or alternatively, processing circuitry 50 may be configured to use machine-learning techniques and/or artificial intelligence to generate separated first source signal and separated second source signal. For example, the circuitry may be configured to implement machine-learning techniques to update the coefficients in a digital filter or in another algorithm. The machine-learning techniques may include frequency-based approaches, wavelet processing approaches, adaptive signal processing approaches, and/or artificial-intelligence-based approaches to generate separated first source signal and separated second source signal, based on sensed differential signals.

In some examples, a first IMD includes a first clock 57 and a second medical device includes a second clock, such as 57 or 157, and processing circuitry 50 is configured to synchronize timing of the first sensed signal and the second sensed signal based on the first and second clocks. For example, processing circuitry 50 may synchronize the first clock and the second clock. The synchronization of the clocks of the different devices may enable processing circuitry 50 to accurately synchronize the first sensed signal and the second sensed signal. For example, a time stamp may be provided on the first sensed signal based on the first clock and a time stamp provided on the second sensed signal based on the second clock, but, in some examples, to synchronize the first sensed signal and the second sensed signal, the first clock and the second clock may need to be synchronized. For example, if the collected first sensed signal and second sensed signal are time stamped with clocks that are not synchronized, the accuracy of the separated source signals may be undermined. Accordingly, in some examples, synchronizing first clock 57 of first IMD 10a with second clock, such as 57 or 157, of second medical device, such as IMD 10b and/or computing device 12b, enables processing circuitry 50 to accurately synchronize the first sensed signal and the second sensed signal, which may enable processing circuitry 50 to accurately separate the first source signal and the second source signal. The separated first source signal and separated second source signal being accurate helps improve the accuracy of a health condition status determined from those signals, which improves the usefulness of the system.

Processing circuitry 50 may determine a health condition status of a patient based at least in part on one or more of the separated first source signal or the separated second source signal. For example, processing circuitry 50 may generate physiological information representative of a health condition status of a patient based on one or more of the separated first source signal or the separated second source signal. In some examples, the health condition status may correspond to one or more of cardiac conditions, seizures, strokes, epilepsy, fainting, neurological conditions, or other medical conditions.

In some examples, the first sensed signal may include a first source signal that is an electrocardiogram ECG signal of the patient and a second source signal that is an ECG signal of a fetus in the patient. In some examples, the second sensed signal may include the first source signal that is an ECG signal of the patient and the second source signal that is as an ECG signal generated by a fetus in the patient. For example, when the first source signal is an ECG signal of a patient and the second source signal in an ECG signal of a fetus in the patient, an ECG signal of the fetus may have a much lower amplitude than an ECG signal of the patient. Processing circuitry 50 performing source separation signal processing, in accordance with the techniques described above, may accurately separate a fetus ECG signal (e.g., second source signal) from the patient ECG signal (e.g., first source signal) so processing circuitry 50 can more accurately determine a health condition of a fetus based on the fetus ECG signal. In some examples, the determined health condition status of the patient may include a health condition status of a fetus in the patient. For example, processing circuitry 50 may be configured to determine the health condition status of the fetus in the patient based on the separated second source signal, which is the ECG signal generated by the fetus.

In some examples, the first sensed signal may include a first source signal that is an atrial activation signal of the patient and a second source signal that is a ventricular activation signal of the patient, and the second sensed signal may include the first source signal that is the atrial activation signal of the patient and the second source signal that is the ventricular activation signal of the patient. In some examples, the first sensed signal may include a first source signal that is an EEG signal of the patient and a second source signal that is an ECG signal of the patient, and the second sensed signal may include the first source signal that is the EEG signal of the patient and the second source signal that is the ECG signal of the patient. In some examples, a first source signal and/or a second source signal may be an ECG signal, EEG signal, EMG signal, electrooculogram (EOG) signal, ventricular activation signal, atrial activation signal, or nerve signal from a patient or a fetus in the patient.

In some examples, processing circuitry 50 may identify a separated source signal, such as the separated first source signal and/or the separated second source signal, based on heuristic techniques based on what is known about the respective sources.

In some examples, processing circuitry 50 is configured to determine a value of a first parameter based on the separated first source signal, determine a value of a second parameter based on the separated second source signal, and determine the health condition status of the patient based at least in part on one or more of the value of the first parameter or the value of the second parameter. In some examples, the first parameter and the second parameter correspond to a same type of physiological parameter, such as an ECG signal (e.g., one ECG signal from mother and one ECG signal from fetus) or an EEG signal. In some examples, processing circuitry 50 may determine a value of the first parameter and or determine a value of the second parameter using heuristic techniques based on what is known about the respective first source and second source.

Patient computing devices 12 are configured for wireless communication with IMDs 10. Computing devices 12 retrieve sensed physiological data from IMDs 10 that was collected and stored by the IMDs 10. In some examples, computing devices 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMDs 10 such as a desktop, laptop, or tablet computer. Computing devices 12 may communicate with IMDs 10 and each other according to the Bluetooth® or Bluetooth® Low Energy (BLE) protocols, as examples. In some examples, only one of computing devices 12, e.g., computing device 12A, is configured for communication with IMDs 10, e.g., due to execution of software (e.g., part of a health monitoring application as described herein) enabling communication and interaction with one or more IMDs 10.

In some examples, computing device(s) 12, e.g., wearable computing device 12B in the example illustrated by FIG. 1, may include electrodes and other sensors to sense physiological signals of patient 4, and may collect and store physiological data and detect episodes based on such signals. Computing device 12B may be incorporated into the apparel of patient 14, such as within clothing, shoes, eyeglasses, a watch or wristband, a hat, etc. In some examples, computing device 12B is a smartwatch or other accessory or peripheral for a smartphone computing device 12A.

One or more of computing devices 12 may be configured to communicate with a variety of other devices or systems via a network 17. For example, one or more of computing devices 12 may be configured to communicate with one or more computing systems, e.g., computing systems 20A and 20B (collectively, “computing systems 20”) via network 17. Computing systems 20A and 20B may be respectively managed by manufacturers of IMDs 10 and computing devices 12 to, for example, provide cloud storage and analysis of collected data, maintenance and software services, or other networked functionality for their respective devices and users thereof. Computing system 20A may comprise, or may be implemented by, the Medtronic Carelink™ Network, in some examples.

Computing device(s) 12 may transmit data, including data retrieved from IMDs 10 to computing system(s) 20 via network 17. The data may include sensed data, e.g., values of physiological parameters measured by IMDs 10 and, in some cases one or more of computing devices 12, data regarding health events detected by IMDs 10 and computing device(s) 12, and other physiological signals or data recorded by IMDs and/or computing device(s) 12.

Network 17 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 17 may include one or more networks administered by service providers, and may thus form part of a large-scale public network infrastructure, e.g., the Internet. Network 17 may provide computing devices and systems, such as those illustrated in FIG. 1, access to the Internet, and may provide a communication framework that allows the computing devices and systems to communicate with one another. In some examples, network 17 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

One or more of IMDs 10 may be configured to transmit data, such as sensed, measured, and/or determined values of physiological parameters (e.g., heart rates, impedance measurements, impedance scores, fluid indices, respiratory rate, activity data, cardiac ECGs, heart sounds, posture, ECG waveform morphological features, ECG waveform morphological features indicating cardiac arrhythmia, QRS morphological features indicating fluid or electrolyte levels, historical physiological data, blood pressure values, etc.), to wireless access points 34 and/or computing device(s) 12. Wireless access points 34 and/or computing device(s) 12 may then communicate the retrieved data to computing systems 20 via network 17.

One or more of IMDs 10 may transmit data over a wired or wireless connection to computing system 20 or to computing device(s) 12. For example, computing device(s) 12 may receive data from IMDs 10 or from computing device(s) 12. In another example, computing device(s) 12 may receive data from computing system 20 or from medical IMDs 10, such as physiological parameter values, diagnostic states, or probability scores, via network 17. In such examples, computing device(s) 12 may determine the data received from computing system 20 or from IMDs 10 and may store the data to a storage device in the computing device(s) accordingly.

In addition, one or more of IMDs 10 may serve as or include data server(s). For example, IMDs 10 may include enough storage capacity or processing power to perform the techniques disclosed herein on a single one of IMDs 10 or on a network of IMDs 10 coordinating tasks via network 17, 31 (e.g., over a private or closed network). In some examples, one of IMDs 10 may include at least one of data server(s). For example, a portable/bedside patient monitor may be able to serve as a data server, as well as serving as one of IMDs 10a, 10b configured to obtain physiological parameter values from patient 4. In other examples, computing system 20 may communicate with each of IMDs 10 via a wired or wireless connection, to receive physiological parameter values or diagnostic states from IMDs 10. In some examples, physiological parameter values may be transferred from IMDs 10 to computing system 20 and/or to computing device(s) 12.

In some cases, computing system 20 may be configured to provide a secure storage site for data that has been collected from IMDs 10 and/or computing device(s) 12. In some instances, computing system 20 may include a database that stores medical- and health-related data. For example, computing system 20 may include a cloud server or other remote server that stores data collected from IMDs 10 and/or computing device(s) 12. In some cases, computing system 20 may assemble data in web pages or other documents for viewing by trained professionals, such as clinicians 39, via clinician computing devices 38. One or more aspects of the example system described with reference to FIG. 2 may be implemented with general network technology and functionality, which may be similar to that provided by the Medtronic CareLink® Network.

In some examples, one or more of clinician computing devices 38 may be a tablet or other smart device located with a clinician, by which the clinician may program, receive alerts from, and/or interrogate IMDs 10. For example, the clinician may access data collected by IMDs 10 through a clinician computing device 38, such as when patient 4 is in between clinician visits, to check on a status of a medical condition. In some examples, the clinician may enter instructions for a medical intervention for patient 4 into an application executed by clinician or other HCP computing device 38, such as based on a status of a patient condition determined by IMDs 10, computing device(s) 12, computing system 20, or any combination thereof, or based on other patient data known to the clinician or other HCP.

One clinician computing device 38 may transmit instructions for medical intervention to another of clinician computing devices 38 located with patient 4 or a caregiver of patient 4. For example, such instructions for medical intervention may include an instruction to change a drug dosage, timing, or selection, to schedule a visit with the clinician, or to seek medical attention. In further examples, a clinician computing device 38 may generate an alert to patient 4 (or relay an alert determined by IMDs 10, computing device(s) 12, or computing system 20) based on a probability score (e.g., posterior probability) determined from physiological parameter values of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

IMD 10a may be implanted outside of a thoracic cavity of patient 4 (e.g., subcutaneously in the pectoral location illustrated in FIG. 1. IMD 10a may be positioned near the sternum near or just below the level of the heart of patient 4, e.g., at least partially within the cardiac silhouette. In some examples, IMD 10a takes the form of the LINQ II™ ICM. IMD 10b may be implanted near or inside the heart. In some examples, IMD 10b takes the form of a pacemaker and may be inserted into the heart. In some examples, IMD 10b may be a leadless pacemaker. In some examples, IMD 10b takes the form of the Micra™, a leadless pacemaker. In some examples, IMD 10b takes the form of a transvenous implantable cardioverter defibrillator (ICD), cardiac resynchronization therapy (CRT) device, an implantable pulse generator (IPG), a subcutaneous ICD (S-ICD), or any other device to measure intracardiac pressures.

Although described primarily in the context of examples in which IMD 10a takes the form of an ICM and IMD 10b takes the form of a pacemaker, the techniques of this disclosure may be implemented in systems including any one or more implantable or external medical devices, including monitors, pacemakers, defibrillators, wearable automatic external defibrillators (WAED), neurostimulators, or drug pumps. In some examples, instead of or in addition to two IMDs 10, system 2 may include a ventricular assist device or WAED in addition to IMD 10a, 10b, 10c.

In some examples, IMD 10a may be configured to receive one or more signals indicative of heart sounds. For example, IMD 10a may be configured to measure heart sounds. IMD 10a may determine fluid index values, such as intravascular fluid index value, using impedance signals received from electrodes and/or measured heart sounds. IMD 10a may also use one or more of the impedance value measurements and the heart sound measurements to determine one or more intravascular fluid index values, impedance scores, and/or various thresholds, such as adaptive thresholds, scoring thresholds, weighting factors for thresholds, and/or cardiac risk thresholds. In some examples, other physiological parameters, such as posture, tissue perfusion, R-wave amplitude and width may also be used to determine one or more intravascular fluid index values.

In some examples, IMD 10a may also sense ECG signals via the plurality of electrodes and/or operate as a therapy delivery device. For example, IMD 10a may additionally operate as a therapy delivery device to deliver electrical signals to the heart of patient 4, such as an implantable pacemaker, a cardioverter, and/or defibrillator, a drug delivery device that delivers therapeutic substances to patient 4 via one or more catheters, or as a combination therapy device that delivers both electrical signals and therapeutic substances.

IMD 10b is configured to measure one or more of intracardiac impedance values and heart sounds, such as A1, A2, A3 and A4 (also referred to as S1, S2, S3 and S4), e.g., amplitude and slew measurements, that indicate intravascular fluid level of patient 4. For example, IMD 10b may be configured to receive one or more signals indicative of intracardiac impedance via intracardiac electrodes of IMD 10b. In some examples, IMD 10b may be a purely diagnostic device. For example, IMD 10b may be a device to measure intracardiac impedance values of patient 4. In some examples, IMD 10b may be configured to receive one or more signals indicative of heart sounds. For example, IMD 10b may be configured to measure heart sounds. IMD 10b may determine fluid index values, such as intravascular fluid index value, using impedance signals received from electrodes and/or measured heart sounds. IMD 10b may also use one or more of the impedance value measurements and the heart sound measurements to determine one or more intravascular fluid index values, impedance scores, and/or various thresholds, such as adaptive thresholds, scoring thresholds, weighting factors for thresholds, and/or cardiac risk thresholds. In some examples, other physiological parameters, such as posture, tissue perfusion, R-wave amplitude and width may also be used to determine one or more intravascular fluid index values.

In some examples, IMD 10b may also sense ECG signals via the plurality of electrodes and/or operate as a therapy delivery device. For example, IMD 10b may additionally operate as a therapy delivery device to deliver electrical signals to the heart of patient 4, such as an implantable pacemaker, a cardioverter, defibrillator, a drug delivery device that delivers therapeutic substances to patient 4 via one or more catheters, and/or as a combination therapy device that delivers both electrical signals and therapeutic substances.

In some examples, IMD 10c may be configured to sense and/or measure features from signals indicative of brain activity and/or cardiac activity. For example, IMD 10c may be configured to sense and/or measure EEG signals via one or more electrodes.

In some examples, the one or more electrodes may be located directly on housing of IMD 10c. In some examples, the one or more electrodes may be located directly within the housing of IMD 10c. In some examples, IMD 10c may also sense and/or measure ECG signals via the one or more electrodes. In some examples, IMD 10c may also operate as a therapy delivery device. For example, IMD 10c may additionally operate as a therapy delivery device to deliver electrical signals to the neck or head of patient 4 and/or deliver electrical signals to tissue inside the head or neck of patient 4. In some examples, IMD 10c may be located at positions such as a rear portion of a patient's 4 neck, a rear portion of the skull, near the patient's 4 temple(s) (e.g., above the ear(s)) and/or over the temporal portion of the skull.

As shown in FIGS. 2, 3, and 5, processing circuitry 50, 130, 23 is located in respective IMD 10a, IMD 10b, patient computing devices 12, and computing systems 20. For simplicity, processing circuitry 50 will be referenced in the examples discussed below, but any one or more of processing circuitries 50, 130, and 23 may be used as the processing circuitry.

In some examples, IMD 10a may obtain sensor data every few minutes, hourly, or even daily. The processing circuitry 50 may aggregate the hourly measurements, while removing noisy measurements. In some examples, IMD 10b may obtain sensor data every few minutes, hourly, or even daily.

In some examples, after processing circuitry 50 detects a health condition status of the patient, as discussed above, IMD 10b may perform treatment in response to the detected health condition status, such as generating electrical impulses to provide pacing therapy, based on the detected health condition status, to cause a heart to contract. IMD 10b may perform one or more various other treatments based on the detected health condition status.

In some examples, after processing circuitry 50 detects a health condition status of the patient, the detected health condition status may be displayed and/or transmitted to another computing device, such as clinician computing device 38, to inform a clinician of the health condition status of patient 4. In some examples, after processing circuitry 50 detects a health condition status of the patient.

As described above, system 2 may be configured to detect health condition status of patient 4, such as cardiac conditions, seizures, strokes, epilepsy, fainting, neurological conditions, or other medical conditions, based on data sensed by IMDs 10a, 10b, or 12B, such as based on at least one or more of the separated first source signal or the separated second source signal, and, in some cases, other data, such as data sensed by computing devices 12A. To detect a status of a health condition, IMDs 10, computing devices 12, and/or computing systems 20 may apply rules to the data, which may be referred to as patient parameter data. In response to detection of a status of a health condition, IMDs 10, computing devices 12, and/or computing systems 20 may wirelessly transmit a message to one or both of computing devices 12 and/or to a clinician computing device 38. The message may indicate the detected health condition status of the patient. The message may indicate a time that IMDs 10 detected the health condition status.

The message may include physiological data collected by IMDs 10, e.g., data which lead to detection of the health condition status, data prior to detection of the health condition status, and/or real-time or more recent data collected after detection of the health condition status. The physiological data may include values of one or more physiological parameters and/or digitized physiological signals such as subcutaneous impedance, intracardiac impedance, interstitial fluid index, intravascular fluid index, interstitial fluid level, intravascular fluid level, tissue oxygen, oxygen saturation, heart rate variability, blood pressure, temperature changes, heart rate changes, fetal heart rate changes, and/or perfusion changes, as well as other physiological parameters. Some examples of statuses of health conditions are cardiac conditions, heart failure (HF), worsening HF, atrial fibrillation, atrial flutter, transient ischemic attack, fetal arrhythmia, chronic obstructive pulmonary disease (COPD), risk of sudden cardiac death (SCD), a ventricular fibrillation, a ventricular tachycardia, myocardial infarction, a pause in heart rhythm (asystole), or Pulseless Electrical Activity (PEA), acute respiratory distress syndrome (ARDS), a stroke, a seizure, or a fall.

Environment 29 includes computing facilities, e.g., a local network 31, by which computing devices 12, and other devices within environment 29 may communicate via network 17. For example, environment 29 may be configured with wireless technology, such as IEEE 802.11 wireless networks, IEEE 802.15 ZigBee networks, an ultra-wideband protocol, near-field communication, or the like. Environment 29 may include one or more wireless access points, e.g., wireless access points 34A and 34B (collectively, “wireless access points 34”) that provide support for wireless communications throughout environment 29. Additionally or alternatively, e.g., when local network is unavailable, computing devices 12, and other devices within environment 29 may be configured to communicate with network 17, via a cellular base station 36 and a cellular network.

In some examples, computing device(s) 12 and/or computing system 20 may implement one or more algorithms to evaluate the sensed physiological data received from IMDs 10. In some examples, computing device(s) 12 and/or computing system 20 may have greater processing capacity than IMDs 10, enabling more complex analysis of the data. In some examples, the computing device(s) 12 and/or computing system 20 may apply the data to a machine learning model or other artificial intelligence developed algorithm, e.g., to determine whether the data is sufficiently indicative of the health condition status.

In some examples, care providers may include emergency medical systems (EMS) and hospitals, and may include particular departments within a hospital, such as an emergency department, catheterization lab, or a stroke response department. Clinician computing devices 38 may include smartphones, desktop, laptop, or tablet computers, or workstations associated with such systems or entities, or employees of such systems or entities. The messages may include any of the data collected by IMDs 10, computing device(s) 12, including sensed physiological data, time of the health condition status, location of patient 4, and results of the analysis by IMDs 10, computing device(s) 12, and/or computing devices 20.

FIG. 2 is a block diagram illustrating an example configuration of IMDs 10 of FIG. 1. IMD 10 may be an example of IMD 10a and/or IMD 10b but will be referenced below as IMD 10 for simplicity. As shown in FIG. 2, IMD 10 may include processing circuitry 50, memory 52, sensing circuitry 54 coupled to electrodes 56A and 56B (hereinafter, “electrodes 56”) and/or one or more sensor(s) 58, clock 57, and/or communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may measure impedance, e.g., of tissue proximate to IMD 10, via electrodes 56. The measured impedance may vary based on respiration, fluid retention, cardiac pulse or flow, and a degree of perfusion or edema. Processing circuitry 50 may determine physiological data relating to respiration, fluid retention, cardiac pulse or flow, perfusion, and/or edema based on the measured impedance.

Sensing circuitry 54 may also monitor signals from electrodes 56 in order to, for example, monitor electrical activity of a heart of patient 4 and produce sensor data for patient 4. In some examples, processing circuitry 50 may identify features of the sensed ECG, such as heart rate, heart rate variability, T-wave alternans, intra-beat intervals (e.g., QT intervals), and/or ECG morphologic features, such as R-wave amplitude, max slew and width, and T-wave morphology, amplitude and slew, to detect an episode of cardiac arrhythmia of patient 4.

In some examples, IMD 10 includes one or more sensors 58, such as one or more accelerometers, gyroscopes, microphones, optical sensors, temperature sensors, pressure sensors, and/or chemical sensors. In some examples, sensing circuitry 52 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52. Patient parameters determined from signals from sensors 58 may include intravascular fluid level, interstitial fluid level, oxygen saturation, glucose level, stress hormone level, heart sounds, body motion, body posture, or blood pressure.

Memory 52 may store applications 70 executable by processing circuitry 50, and data 80. Applications 70 may include a health condition status surveillance application 72. Processing circuitry 50 may execute status surveillance application 72 to detect a health condition status of patient 4 based on combination of one or more of the types of physiological data from IMD 10a and/or IMD 10b, described herein, which may be stored as sensed data 82. In some examples, sensed data 82 may additionally include patient parameter data sensed by other devices, e.g., computing device(s) 12, and received via communication circuitry 60. Status surveillance application 72 may be configured with a rules engine 74. Rules engine 74 may apply rules 84 to sensed data 82. Rules 84 may include one or more models, algorithms, decision trees, and/or thresholds. In some cases, rules 84 may be developed based on machine learning, e.g., may include one or more machine learning models.

As examples, status surveillance application 72 may detect cardiac conditions, HF, worsening HF, COPD, risk of SCD, sudden cardiac arrest (SCA), a ventricular fibrillation, a ventricular tachycardia, supra-ventricular tachycardia (e.g., conducted atrial fibrillation), ventricular asystole, or a myocardial infarction based on an ECG and/or other patient parameter data indicating the electrical or mechanical activity of the heart of patient 4. In some examples, status surveillance application 72 may detect stroke based on such cardiac activity data. In some examples, sensing circuitry 54 may detect brain activity data, e.g., an electroencephalogram (EEG) via electrodes 56, and status surveillance application 72 may detect stroke or a seizure based on the brain activity alone, or in combination with cardiac activity data or other physiological data, such as accelerometer data. For example, accelerometer data that indicates activity data and/or posture data of patient 4. When status surveillance application 72 detects a status of a health condition, status surveillance application 72 may store the sensed data 82 that lead to the detection (and in some cases a window of data preceding and/or following the detection) as event data 86.

In some examples, in response to detection of a health condition status, processing circuitry 50 transmits, via communication circuitry 60, event data 86 for the event to computing device(s) 12 (FIG. 1). This transmission may be included in a message indicating the health condition status, as described herein. Transmission of the message may occur on an ad hoc basis and as quickly as possible. Communication circuitry 60 may include any suitable hardware, firmware, software, or any combination thereof for wirelessly communicating with another device, such as computing devices 12, and/or other IMD 10.

In some examples, communication circuitry 60 in IMD 10a may communicate with IMD 10b to share information such as sensed data 82 and/or event data 86 to be used in the detecting a status of a health condition of patient 4. In some examples, IMD 10b may also communicate with IMD 10A to share information such as sensed data 82 and/or event data 86 to be used in the detecting a status of a health condition of patient 4.

FIG. 3 is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a laptop, a tablet computer, a personal digital assistant (PDA), a smartwatch or other wearable computing device. In some examples, computing devices 38 and 42 may be configured similarly to the configuration of computing device 12 illustrated in FIG. 3.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, one or more sensors 138, clock 157, and/or communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure.

Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random access memories (DRAM), static random access memories (SRAM), and other forms of volatile memories known in the art. Memory 132, in some examples, also includes one or more memories configured for long-term storage of information, e.g. including non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In some examples, memory 132 includes cloud-associated storage.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, haptic, audio, and visual output. Output devices 134 of computing device 12 may include a presence-sensitive screen, sound card, video graphics adapter card, speaker, cathode ray tube monitor, liquid crystal display (LCD), light emitting diodes (LEDs), or any type of device for generating tactile, audio, and/or visual output.

One or more sensors 138 of computing device 12 may sense physiological parameters or signals of patient 4. Sensor(s) 138 may include electrodes, accelerometers (e.g., 3-axis accelerometers), an optical sensor, impedance sensors, temperature sensors, pressure sensors, heart sound sensors (e.g., microphones), and other sensors, and sensing circuitry (e.g., including an ADC), similar to those described above with respect to IMDs 10 and FIG. 2.

Communication circuitry 140 of computing device 12 may communicate with other devices by transmitting and receiving data. Communication circuitry 140 may receive data from IMDs 10, such as subcutaneous impedance and intracardiac impedance, from communication circuitry in IMDs 10. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 3, health monitoring application 150 executes in user space 102 of computing device 12. Health monitoring application 150 may be logically divided into presentation layer 152, application layer 154, and data layer 156. Presentation layer 152 may include a user interface (UI) component 160, which generates and renders user interfaces of health monitoring application 150.

Application layer 154 may include, but is not limited to, status engine 170, rules engine 172, rules configuration component 174, status assistant 176, and location service 178. Status engine 170 may be responsive to receipt of a transmission from IMDs 10 indicating that sensed data 190 from IMDs 10 has been received and begin a determination of a health condition status of patient 4. Status engine 170 may also control performance of any of the operations in response to detection of a health condition status ascribed herein to computing device 12.

Patient input 192 may include responses to queries posed by health monitoring application 150 regarding the condition of patient 4, input by patient 4 or another user. The queries and responses may occur responsive to the detection of the health condition status by IMDs 10 or may have occurred prior to the detection, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition or treatments of patient 4 described above. EHR data 194 may relate to history of SCA, tachyarrhythmia, myocardial infarction, stroke, seizure, one or more disease states, such as status of heart failure, COPD, renal dysfunction, or hypertension, aspects of disease state, such as ECG characteristics, EEG characteristics, cardiac ischemia, oxygen saturation, lung fluid, activity, or metabolite level, genetic conditions, congenital anomalies, history of procedures, such as ablation or cardioversion, and healthcare utilization. EHR data 194 may also include intracranial pressure indicators or cardiac indicators, such as ejection fraction and left-ventricular wall thickness. EHR data 194 may also include demographic and other information of patient 4, such as age, gender, race, height, weight, and BMI.

Rules engine 172 may apply rules 196 to the data. Rules 196 may include one or more models, algorithms, decision trees, and/or thresholds. The rules 196 may include any of the rules discussed above with respect to a relationship of interstitial fluid levels and intravascular fluid levels to determining a health condition status. The rules 196 may further include additional parameters measured by any of the sensors discussed above.

In some cases, rules 196 may be developed based on machine learning, e.g., may include one or more machine learning models. In some examples, rules 196 and the operation of rules engine 172 may provide a more complex analysis the patient parameter data, e.g., the data received from IMDs 10, than is provided by rules engine 74 and rules 84. In examples in which rules 196 include one or more machine learning models, rules engine 172 may apply feature vectors derived from the data to the model(s).

Rules configuration component 174 may be configured to modify rules 196 (and in some examples rules 84) based on feedback indicating whether the detections and confirmations of statuses of health conditions by IMDs 10 and/or computing device 12 were accurate. The feedback may be received from patient 4, or from clinicians 39. In some examples, rules configuration component 174 may utilize the data sets from true and false detections and confirmations for supervised machine learning to further train models included as part of rules 196.

Rules configuration component 174, or another component executed by processing circuitry of system 2, may select a configuration of rules 196 based on etiological data for patient, e.g., any combination of one or more of the examples of sensed data 190, patient input 192, and EHR data 194 discussed above. In some examples, different sets of rules 196 tailored to different cohorts of patients may be available for selection for patient 4 based on such etiological data.

As discussed above, status assistant 176 may provide a conversational interface for patient 4 to exchange information with computing device 12. Responses from the user may be included as patient input 192. Status assistant 176 may use natural language processing and context data to interpret utterances by the user. In some examples, in addition to receiving responses to queries posed by the assistant, status assistant 176 may be configured to respond to queries posed by the user. In some examples, status assistant 176 may provide directions to and respond to queries regarding treatment of patient 4.

Location service 178 may determine the location of computing device 12 and, thereby, the presumed location of patient 4. Location service 178 may use global position system (GPS) data, multilateration, and/or any other known techniques for locating computing devices.

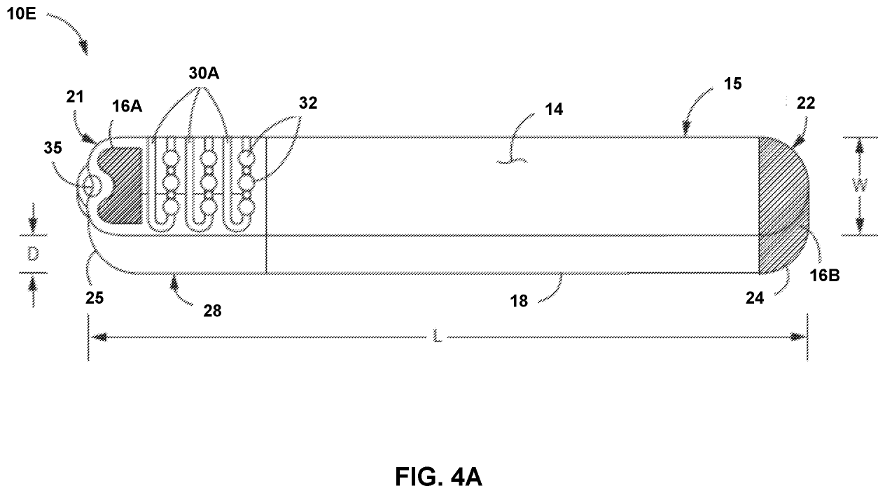

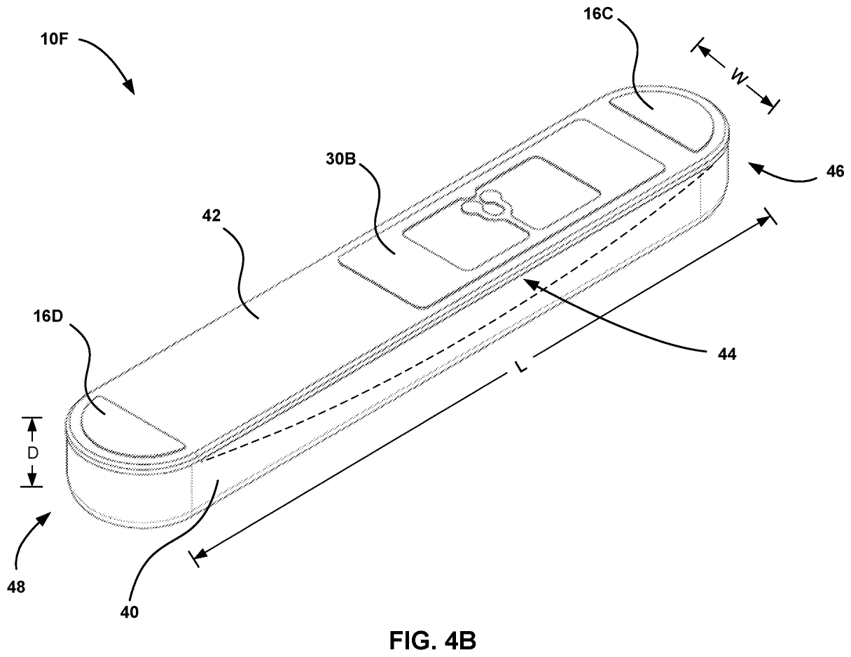

FIG. 4A is a conceptual drawing illustrating an IMD 10E, which may be an example configuration of IMD 10 of FIG. 2 as an ICM. In the example shown in FIG. 4A, IMD 10E may be embodied as a monitoring device having housing 15, proximal electrode 16A and distal electrode 16B. Housing 15 may further comprise first major surface 14, second major surface 18, proximal end 21, and distal end 22. Housing 15 encloses electronic circuitry located inside the IMD 10E and protects the circuitry contained therein from body fluids. Electrical feedthroughs provide electrical connection of electrodes 16A and 16B.

In the example shown in FIG. 4A, IMD 10E is defined by a length L, a width W and thickness or depth D and is in the form of an elongated rectangular prism wherein the length L is much larger than the width W, which in turn is larger than the depth D. In one example, the geometry of the IMD 10E—in particular a width W greater than the depth D—is selected to allow IMD 10E to be inserted under the skin of the patient using a minimally invasive procedure and to remain in the desired orientation during insertion. For example, the device shown in FIG. 4A includes radial asymmetries (notably, the rectangular shape) along the longitudinal axis that maintains the device in the proper orientation following insertion. For example, the spacing between proximal electrode 64 and distal electrode 66 may range from 30 millimeters (mm) to 55 mm, 35 mm to 55 mm, and from 40 mm to 55 mm and may be any range or individual spacing from 25 mm to 60 mm. In addition, IMD 10E may have a length L that ranges from 30 mm to about 70 mm. In other examples, the length L may range from 5 mm to 60 mm, 15 mm to 50 mm, 40 mm to 60 mm, 45 mm to 60 mm and may be any length or range of lengths between about 5 mm and about 80 mm. In addition, the width W of major surface 14 may range from 5 mm to 15 mm, 3 mm to 10 mm, and may be any single or range of widths between 3 mm and 15 mm. The thickness of depth D of IMD 10E may range from 2 mm to 9 mm. In other examples, the depth D of IMD 10E may range from 2 mm to 5 mm, may range from 5 mm to 15 mm, and may be any single or range of depths from 2 mm to 15 mm. In addition, IMD 10A according to an example of the present disclosure has a geometry and size designed for ease of implant and patient comfort. Examples of IMD 10E described in this disclosure may have a volume of three cubic centimeters (cm) or less, 1.5 cubic cm or less or any volume between three and 1.5 cubic centimeters.

In the example shown in FIG. 4A, once inserted within the patient, the first major surface 14 faces outward, toward the skin of the patient while the second major surface 18 is located opposite the first major surface 14. In addition, in the example shown in FIG. 4A, proximal end 21 and distal end 22 are rounded to reduce discomfort and irritation to surrounding tissue once inserted under the skin of the patient. IMD 10E, including instrument and method for inserting IMD 10 is described, for example, in U.S. Patent Publication No. 2014/0276928, incorporated herein by reference in its entirety.

Proximal electrode 16A and distal electrode 16B are used to sense cardiac signals, e.g. ECG signals, intra-thoracically or extra-thoracically, which may be sub-muscularly or subcutaneously. ECG signals may be stored in a memory of IMD 10E, and data may be transmitted via integrated antenna 30A to another medical device, which may be another implantable device or an external device, such as external device 12. In some examples, electrodes 16A and 16B may additionally or alternatively be used for sensing any bio-potential signal of interest, which may be, for example, an ECG, EEG, EMG, EOG, or a nerve signal, from any implanted location.

In the example shown in FIG. 4A, proximal electrode 16A is in close proximity to the proximal end 21 and distal electrode 16B is in close proximity to distal end 22. In this example, distal electrode 16B is not limited to a flattened, outward facing surface, but may extend from first major surface 14 around rounded edges 24 and/or end surface 25 and onto the second major surface 18 so that the electrode 16B has a three-dimensional curved configuration. In some examples, electrode 16B is an uninsulated portion of a metallic, e.g., titanium, part of housing 15.

In the example shown in FIG. 4A, proximal electrode 16A is located on first major surface 14 and is substantially flat, and outward facing. However, in other examples proximal electrode 16A may utilize the three dimensional curved configuration of distal electrode 16B, providing a three dimensional proximal electrode (not shown in this example). Similarly, in other examples distal electrode 16B may utilize a substantially flat, outward facing electrode located on first major surface 14 similar to that shown with respect to proximal electrode 16A.

The various electrode configurations allow for configurations in which proximal electrode 16A and distal electrode 16B are located on both first major surface 14 and second major surface 18. In other configurations, such as that shown in FIG. 4A, only one of proximal electrode 16A and distal electrode 16B is located on both major surfaces 14 and 18, and in still other configurations both proximal electrode 16A and distal electrode 16B are located on one of the first major surface 14 or the second major surface 18 (e.g., proximal electrode 16A located on first major surface 14 while distal electrode 16B is located on second major surface 18). In another example, IMD 10E may include electrodes on both major surface 14 and 18 at or near the proximal and distal ends of the device, such that a total of four electrodes are included on IMD 10E. Electrodes 16A and 16B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 4A, proximal end 21 includes a header assembly 28 that includes one or more of proximal electrode 16A, integrated antenna 30A, anti-migration projections 32, and/or suture hole 35. Integrated antenna 30A is located on the same major surface (i.e., first major surface 14) as proximal electrode 16A and is also included as part of header assembly 28. Integrated antenna 30A allows IMD 10E to transmit and/or receive data. In other examples, integrated antenna 30A may be formed on the opposite major surface as proximal electrode 16A, or may be incorporated within the housing 15 of IMD 10E. In the example shown in FIG. 4A, anti-migration projections 32 are located adjacent to integrated antenna 30A and protrude away from first major surface 14 to prevent longitudinal movement of the device. In the example shown in FIG. 4A, anti-migration projections 32 include a plurality (e.g., nine) small bumps or protrusions extending away from first major surface 14. As discussed above, in other examples anti-migration projections 32 may be located on the opposite major surface as proximal electrode 16A and/or integrated antenna 30A. In addition, in the example shown in FIG. 4A, header assembly 28 includes suture hole 35, which provides another means of securing IMD 10E to the patient to prevent movement following insertion. In the example shown, suture hole 35 is located adjacent to proximal electrode 16A. In one example, header assembly 28 is a molded header assembly made from a polymeric or plastic material, which may be integrated or separable from the main portion of IMD 10E.

FIG. 4B is a perspective drawing illustrating another IMD 10F, which may be another example configuration of IMD 10 from FIG. 2. IMD 10F of FIG. 4B may be configured substantially similarly to IMD 10F of FIG. 4A, with differences between them discussed herein.

IMD 10F may include a leadless, subcutaneously-implantable monitoring device, e.g. an ICM. IMD 10F includes housing having a base 40 and an insulative cover 42. Proximal electrode 16C and distal electrode 16D may be formed or placed on an outer surface of cover 42. Various circuitries and components of IMD 10F may be formed or placed on an inner surface of cover 42, or within base 40. In some examples, a battery or other power source of IMD 10F may be included within base 40. In the illustrated example, antenna 30B is formed or placed on the outer surface of cover 42, but may be formed or placed on the inner surface in some examples. In some examples, insulative cover 42 may be positioned over an open base 40 such that base 40 and cover 42 enclose the circuitries and other components and protect them from fluids such as body fluids.

Circuitries and components may be formed on the inner side of insulative cover 42, such as by using flip-chip technology. Insulative cover 42 may be flipped onto a base 40. When flipped and placed onto base 40, the components of IMD 10F formed on the inner side of insulative cover 42 may be positioned in a gap 44 defined by base 40.

Electrodes 16C and 16D and antenna 30B may be electrically connected to circuitry formed on the inner side of insulative cover 42 through one or more vias (not shown) formed through insulative cover 42. Insulative cover 42 may be formed of sapphire (i.e., corundum), glass, parylene, and/or any other suitable insulating material. Base 40 may be formed from titanium or any other suitable material (e.g., a biocompatible material). Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 4B, the housing of IMD 10F defines a length L, a width W and thickness or depth D and is in the form of an elongated rectangular prism wherein the length L is much larger than the width W, which in turn is larger than the depth D, similar to IMD 10E of FIG. 4A. For example, the spacing between proximal electrode 64 and distal electrode 66 may range from 30 millimeters (mm) to 50 mm, from 35 mm to 45 mm, or be approximately 40 mm. In addition, IMD 10F may have a length L that ranges from 30 mm to about 70 mm. In other examples, the length L may range from 5 mm to 60 mm, 40 mm to 60 mm, 45 mm to 55 mm, or be approximately 45 mm. In addition, the width W may range from 3 mm to 15 mm, such as approximately 8 mm. The thickness of depth D of IMD 10F may range from 2 mm to 15 mm, from 3 to 5 mm, or be approximately 4 mm. IMD 10F may have a volume of three cubic centimeters (cm) or less, or 1.5 cubic cm or less, such as approximately 1.4 cubic cm.

In the example shown in FIG. 4B, once inserted subcutaneously within the patient, outer surface of cover 42 faces outward, toward the skin of the patient. In addition, as shown in FIG. 4B, proximal end 46 and distal end 48 are rounded to reduce discomfort and irritation to surrounding tissue once inserted.

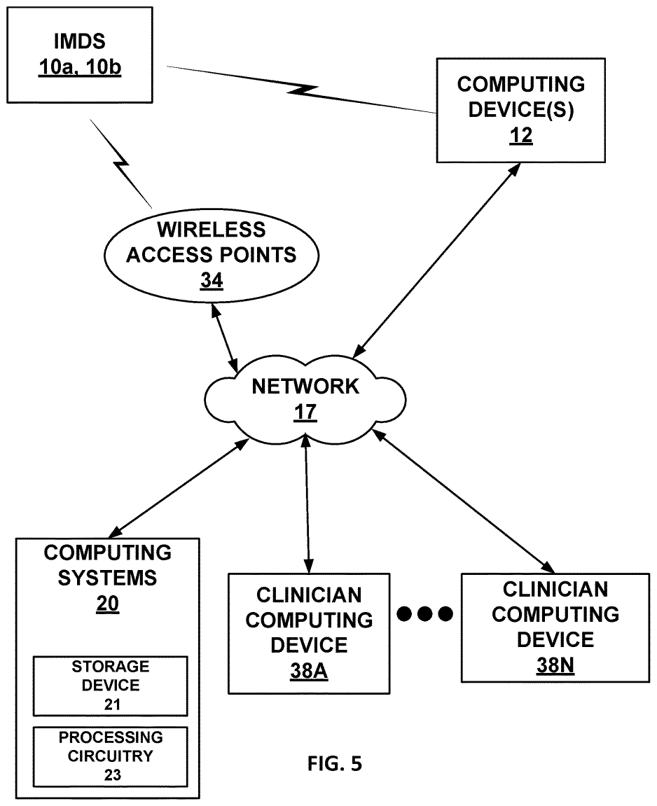

FIG. 5 is a block diagram illustrating an example system that includes wireless access points 34, a network 17, external computing devices, such as computing systems 20, and one or more other clinician computing devices 38A-38N (collectively, “clinician computing devices 38”), which may be coupled to IMDs 10 and computing device(s) 12 via network 17, in accordance with one or more techniques described herein. In this example, IMDs 10 may use communication circuitry 60 to communicate with computing device(s) 12 via a first wireless connection, and to communicate with wireless access points 34 via a second wireless connection. In the example of FIG. 5, wireless access points 34, computing device(s) 12, computing systems 20, and clinician computing devices 38 are interconnected and may communicate with each other through network 17. Network 17 may include a local area network, wide area network, or global network, such as the Internet. The system of FIG. 5 may be implemented, in some aspects, with general network technology and functionality similar to that provided by the Medtronic CareLink® Network.

Wireless access points 34 may include a device that connects to network 17 via any of a variety of connections, such as telephone dial-up, digital subscriber line (DSL), or cable modem connections. In other examples, wireless access points 34 may be coupled to network 17 through different forms of connections, including wired or wireless connections. In some examples, wireless access points 34 may be a user device, such as a tablet or smartphone, that may be co-located with the patient. IMD 10 may be configured to transmit data, such as impedance value information, impedance scores, and/or ECGs, to wireless access points 34. Wireless access points 34 may then communicate the retrieved data to computing systems 20 via network 17.

In some cases, computing systems 20 may be configured to provide a secure storage site for data that has been collected from IMDs 10 and/or computing device(s) 12. In some cases, computing systems 20 may assemble data in web pages or other documents for viewing by trained professionals, such as clinicians, via clinician computing devices 38. One or more aspects of the illustrated system of FIG. 5 may be implemented with general network technology and functionality, which may be similar to that provided by the Medtronic CareLink® Network.

In some examples, computing systems 20 may monitor impedance, e.g., based on measured impedance information received from IMDs 10 and/or computing device(s) 12 via network 17, to detect worsening heart failure of patient 4 using any of the techniques described herein. Computing systems 20 may provide alerts relating to worsening heart failure of patient 4 via network 17 to patient 4 via wireless access points 34, or to one or more clinicians via computing devices 100. In examples such as those described above in which IMD 10 and/or computing device(s) 12 monitor the impedance, computing systems 20 may receive an alert from IMDs 10 or computing device(s) 12 via network 17, and provide alerts to one or more clinicians via clinician computing devices 38. In some examples, computing systems 20 may generate web-pages to provide alerts and information regarding the impedance, and may include a memory to store alerts and diagnostic or physiological parameter information for a plurality of patients.

In some examples, one or more of clinician computing devices 38 may be a tablet or other smart device located with a clinician, by which the clinician may program, receive alerts from, and/or interrogate IMDs 10. For example, the clinician may access data collected by IMDs 10 through a clinician computing device 38, such as when patient 4 is in between clinician visits, to check on a status of a medical condition. In some examples, the clinician may enter instructions for a medical intervention for patient 4 into an application executed by clinician computing device 38, such as based on a status of a patient condition determined by IMDs 10, computing device(s) 12, computing systems 20, or any combination thereof, or based on other patient data known to the clinician. Clinician computing device 100 then may transmit the instructions for medical intervention to another of clinician computing devices 100 located with patient 4 or a caregiver of patient 4.

In some examples, instructions for medical intervention may include an instruction to change a drug dosage, timing, or selection, to schedule a visit with the clinician, or to seek medical attention. In further examples, a clinician computing device 38 may generate an alert to patient 4 based on a status of a medical condition of patient 4, which may enable patient 4 to proactively seek medical attention prior to receiving instructions for a medical intervention. In this manner, patient 4 may be empowered to take action, as needed, to address his or her medical status, which may help improve clinical outcomes for patient 4.

In the example illustrated by FIG. 5, computing system 20 includes a storage device 21, e.g., to store data retrieved from IMDs 10, and processing circuitry 23. Although not illustrated in FIG. 5 clinician computing devices 38 may similarly include a storage device and processing circuitry. Processing circuitry 23 may include one or more processors that are configured to implement functionality and/or process instructions for execution within computing systems 20. For example, processing circuitry 23 may be capable of processing instructions stored in storage device 21. Processing circuitry 23 may include, for example, microprocessors, DSPs, ASICs, FPGAs, or equivalent discrete or integrated logic circuitry, or a combination of any of the foregoing devices or circuitry. Accordingly, processing circuitry 23 may include any suitable structure, whether in hardware, software, firmware, or any combination thereof, to perform the functions ascribed herein to processing circuitry 98. Processing circuitry 23 of computing systems 20 and/or the processing circuitry of clinician computing devices 38 may implement any of the techniques described herein to analyze impedance values received from IMD 10, e.g., to determine a health condition status of patient 4 (e.g., worsening heart failure).

Storage device 21 may include a computer-readable storage medium or computer-readable storage device. In some examples, storage device 21 includes one or more of a short-term memory or a long-term memory. Storage device 21 may include, for example, RAM, DRAM, SRAM, magnetic discs, optical discs, flash memories, or forms of EPROM or EEPROM. In some examples, storage device 21 is used to store data indicative of instructions for execution by processing circuitry 23.

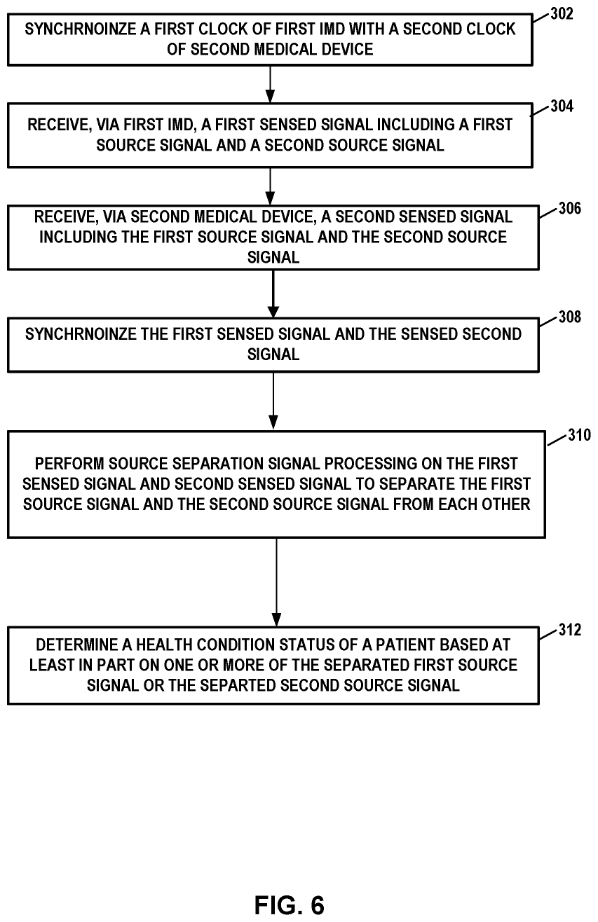

FIG. 6 is a flow diagram illustrating an example operation of determining a health condition of a patient, in accordance with one or more techniques of this disclosure. The example operation of FIG. 6 may be performed by processing circuitry of any one of IMDs 10, computing device(s) 12, computing systems 20, or clinician computing devices 38 (e.g., by processing circuitry 50, 130, or 23), or by processing circuitry of two or more of these devices respectively performing portions of the example operation.

As seen in the example of FIG. 6, processing circuitry may synchronize a first clock 57 of first IMD 10a with a second clock 57 or 157 of a second medical device 10b or 12B (302). Next, processing circuitry may receive, via first IMD, a first sensed signal including a first source signal and a second source signal and may receive, via second medical device, a second sensed signal including the first source signal and the second source signal (306). Processing circuitry may then synchronize the first sensed signal and the second sensed signal (308). In some examples, processing circuitry may confirm the first sensed signal and the second sensed signal are synchronized, e.g., due to the first clock and second clock being synchronized. Processing circuitry may then perform source separation signal processing, such as BSS, on the first sensed signal and second sensed signal to separate the first source signal and the second source signal from each other (310). Processing circuitry may then determine a health condition status of a patient based at least in part on one or more of the separated first source signal or the separated second source signal (312).

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1: A system includes a first implantable medical device (IMD) includes receive the first sensed signal and the second sensed signal; synchronize timing of the first sensed signal and the second sensed signal; perform source separation signal processing on the first sensed signal and second sensed signal to separate the first source signal and the second source signal from each other; and determine a health condition status of a patient based at least in part on one or more of the separated first source signal or the separated second source signal.

Example 2: The system of example 1, wherein the first IMD includes a first clock, the second medical device includes a second clock, and the processing circuitry is further configured to synchronize the first clock and the second clock.

Example 3: The system of any of examples 1-2, wherein the processing circuitry is further configured to: determine a value of a first parameter based on the separated first source signal; determine a value of a second parameter based on the separated second source signal; and determine the health condition status of the patient based at least in part on one or more of the value of the first parameter or the value of the second parameter.

Example 4: The system of example 3, wherein the first parameter and the second parameter correspond to a same type of physiological parameter.

Example 5: The system of any of examples 1-4, wherein the first IMD is configured to be positioned in a first location of a body of the patient and the second medical device is configured to be positioned in a second location, the first location being different than the second location.

Example 6: The system of example 5, wherein the first location is proximal to a heart of the patient or a head of the patient.

Example 7: The system of any of examples 5-6, wherein the second location is proximal to a fetus in the patient, a head of the patient, or external to the patient.

Example 8: The system of any of examples 1-7, wherein the first source signal is an electrocardiogram (ECG) signal of the patient.

Example 9: The system of example 8, wherein the second source signal is an ECG signal generated by a fetus in the patient.

Example 10: The system of example 9, wherein the determined health condition status of the patient is a health condition status of the fetus in the patient, and the processing circuitry is configured to determine the health condition status of the fetus in the patient based on the separated second source signal.

Example 11: The system of any of examples 1-10, wherein the first IMD is an implantable cardiac monitor (ICM) includes a power source operatively coupled to the processing circuitry; a memory operatively coupled to the processing circuitry; a distal electrode operatively coupled to the processing circuitry; a proximal electrode operatively coupled to the processing circuitry; and a hermetically-sealed housing configured for subcutaneous implantation within the patient, wherein at least the power source, the memory, and the processing circuitry are within the hermetically-sealed case, and wherein the housing has a length, a width, and a depth, wherein the length is greater than the width and the width is greater than the depth, wherein the length is within a range from 5 millimeters (mm) to 60 mm, wherein the width is within a range from 5 mm to 15 mm, and wherein the depth is within a range from 5 mm to 15 mm.

Example 12: The system of any of examples 1-11, wherein the second medical device is an IMD.

Example 13: A method for operating processing circuitry of a computing device includes receiving, by the processing circuitry and via a first implantable medical device (IMD) implanted in a patient, a first sensed signal including a first source signal and a second sensed signal; receiving, by the processing circuitry and via a second medical device coupled to the patient, a second sensed signal including the first source signal and the second source signal; synchronizing, by the processing circuitry, timing of the first sensed signal and the second sensed signal; performing, by the processing circuitry, source separation signal processing on the first sensed signal and second sensed signal to separate the first source signal and the second source signal from each other; and determining, by the processing circuitry, a health condition status of the patient based at least in part on one or more of the separated first source signal or the separated second source signal.

Example 14: The method of example 13, the method further comprising synchronizing, by the processing circuitry, a first clock of the first IMD with a second clock of the second medical device.

Example 15: The method of any of examples 13-14, the method further includes determining, by the processing circuitry, a value of a first parameter based on the separated first source signal; determining, by the processing circuitry, a value of a second parameter based on the separated second source signal; and determining, by the processing circuitry, the health condition status of the patient based at least in part on one or more of the value of the first parameter or the value of the second parameter.

Example 16: The method of example 15, wherein the first parameter and the second parameter correspond to a same type of physiological parameter.

Example 17: The method of any of examples 13-16, wherein the first IMD is configured to be positioned in a first location of a body of the patient and the second medical device is configured to be positioned in a second location, the first location being different than the second location.

Example 18: The method of example 17, wherein the first location is proximal to a heart of the patient or a head of the patient.

Example 19: The method of any of examples 17-18, wherein the second location is proximal to a fetus in the patient, a head of the patient, or external to the patient.

Example 20: The method of any of examples 13-19, wherein the first source signal is an electrocardiogram (ECG) signal of the patient.

Example 21: The method of example 20, wherein the second source signal is an ECG signal generated by a fetus in the patient.

Example 22: The method of example 21, wherein the determined health condition status of the patient is a health condition status of the fetus in the patient.

Example 23: The method of example 22, the method further comprising determining the health condition status of the fetus in the patient based on the separated second source signal.

Example 24: The method of any of examples 13-23, wherein the first IMD is an implantable cardiac monitor (ICM).

Example 25: The method of any of examples 13-24, wherein the second medical device is an IMD.

Example 26: A computing device includes a memory; and processing circuitry coupled to the memory; the processing circuitry being configured to: receive, via a first implantable medical device (IMD), a first sensed signal including a first source signal and a second source signal; receive, via a second medical device, a second sensed signal including the first source signal and the second source signal; synchronize timing of the first sensed signal and the second sensed signal; perform source separation signal processing on the first sensed signal and second sensed signal to separate the first source signal and the second source signal from each other; and determine the health condition status of the patient based at least in part on one or more of the separated first source signal or the separated second source signal.

Example 27: The computing device of example 26, wherein the processing circuitry is further configured to synchronize a first clock of the first IMD and a second clock of the second medical device.

Example 28: The computing device of any of examples 26-27, wherein the processing circuitry is further configured to: determine a value of a first parameter based on the separated first source signal; determine a value of a second parameter based on the separated second source signal; and determine the health condition status of the patient based at least in part on one or more of the value of the first parameter or the value of the second parameter.

Example 29: The computing device of example 28, wherein the first parameter and the second parameter correspond to a same type of physiological parameter.

Example 30: The computing device of any of examples 26-29, wherein the first IMD is positioned in a first location of a body of the patient and the second medical device is positioned in a second location, the first location being different than the second location.

Example 31: The computing device of example 30, wherein the first location is proximal to a heart of the patient or a head of the patient.

Example 32: The computing device of any of examples 30-31, wherein the second location is proximal to a fetus in the patient, a head of the patient, or external to the patient.

Example 33: The computing device of any of examples 26-32, wherein the first source signal is an electrocardiogram (ECG) signal of the patient.

Example 34: The computing device of example 33, wherein the second source signal is an ECG signal generated by a fetus in the patient.

Example 35: The computing device of example 34, wherein the determined health condition status of the patient is a health condition status of the fetus in the patient, and the processing circuitry is further configured to determine the health condition status of the fetus in the patient based on the separated second source signal.

Example 36: The computing device of any of examples 26-35, wherein the first IMD is an implantable cardiac monitor (ICM).

Example 37: The computing device of any of examples 26-36, wherein the second medical device is an IMD.

Various examples have been described. These and other examples are within the scope of the following claims.

1. A system comprising:
a first implantable medical device (IMD) comprising one or more first sensors, the first IMD configured to receive a first sensed signal from the one or more first sensors, the first sensed signal including a first source signal and a second source signal;
a second medical device comprising one or more second sensors, the second medical device configured to receive a second sensed signal from the one or more second sensors, the second sensed signal including the first source signal and the second source signal; and
processing circuitry configured to:
receive the first sensed signal and the second sensed signal;
synchronize timing of the first sensed signal and the second sensed signal;
perform source separation signal processing on the first sensed signal and second sensed signal to separate the first source signal and the second source signal from each other; and
determine a health condition status of a patient based at least in part on one or more of the separated first source signal or the separated second source signal.

2. The system of claim 1, wherein the first IMD includes a first clock, the second medical device includes a second clock, and the processing circuitry is further configured to synchronize the first clock and the second clock.

3. The system of claim 1, wherein the processing circuitry is further configured to:
determine a value of a first parameter based on the separated first source signal;
determine a value of a second parameter based on the separated second source signal; and
determine the health condition status of the patient based at least in part on one or more of the value of the first parameter or the value of the second parameter.

4. The system of claim 3, wherein the first parameter and the second parameter correspond to a same type of physiological parameter.

5. The system of claim 1, wherein the first IMD is configured to be positioned in a first location of a body of the patient and the second medical device is configured to be positioned in a second location, the first location being different than the second location.

6. The system of claim 1, wherein the first source signal is an electrocardiogram (ECG) signal of the patient.

7. The system of claim 6, wherein the second source signal is an ECG signal generated by a fetus in the patient.

8. The system of claim 7, wherein the determined health condition status of the patient is a health condition status of the fetus in the patient, and the processing circuitry is configured to determine the health condition status of the fetus in the patient based on the separated second source signal.

9. The system of claim 1, wherein the first IMD is an implantable cardiac monitor (ICM) comprising:
a power source operatively coupled to the processing circuitry;
a memory operatively coupled to the processing circuitry;
a distal electrode operatively coupled to the processing circuitry;
a proximal electrode operatively coupled to the processing circuitry; and
a hermetically-sealed housing configured for subcutaneous implantation within the patient, wherein at least the power source, the memory, and the processing circuitry are within the hermetically-sealed case, and
wherein the housing has a length, a width, and a depth,
wherein the length is greater than the width and the width is greater than the depth,
wherein the length is within a range from 5 millimeters (mm) to 60 mm,
wherein the width is within a range from 5 mm to 15 mm, and
wherein the depth is within a range from 5 mm to 15 mm.

10. A computing device comprising:
a memory; and
processing circuitry coupled to the memory; the processing circuitry being configured to:
receive, via a first implantable medical device (IMD), a first sensed signal including a first source signal and a second source signal;
receive, via a second medical device, a second sensed signal including the first source signal and the second source signal;
synchronize timing of the first sensed signal and the second sensed signal;
perform source separation signal processing on the first sensed signal and second sensed signal to separate the first source signal and the second source signal from each other; and
determine the health condition status of the patient based at least in part on one or more of the separated first source signal or the separated second source signal.

11. The computing device of claim 10, wherein the processing circuitry is further configured to synchronize a first clock of the first IMD and a second clock of the second medical device.

12. The computing device of claim 10, wherein the processing circuitry is further configured to:
determine a value of a first parameter based on the separated first source signal;
determine a value of a second parameter based on the separated second source signal; and
determine the health condition status of the patient based at least in part on one or more of the value of the first parameter or the value of the second parameter.

13. The computing device of claim 12, wherein the first parameter and the second parameter correspond to a same type of physiological parameter.

14. The computing device of claim 10, wherein the first IMD is positioned in a first location of a body of the patient and the second medical device is positioned in a second location, the first location being different than the second location.

15. The computing device of claim 14, wherein the first location is proximal to a heart of the patient or a head of the patient and the second location is proximal to a fetus in the patient, a head of the patient, or external to the patient.

16. A method for operating processing circuitry of a computing device, the method comprising:
receiving, by the processing circuitry and via a first implantable medical device (IMD) implanted in a patient, a first sensed signal including a first source signal and a second sensed signal;
receiving, by the processing circuitry and via a second medical device coupled to the patient, a second sensed signal including the first source signal and the second source signal;
synchronizing, by the processing circuitry, timing of the first sensed signal and the second sensed signal;
performing, by the processing circuitry, source separation signal processing on the first sensed signal and second sensed signal to separate the first source signal and the second source signal from each other; and
determining, by the processing circuitry, a health condition status of the patient based at least in part on one or more of the separated first source signal or the separated second source signal.

17. The method of claim 16, the method further comprising synchronizing, by the processing circuitry, a first clock of the first IMD with a second clock of the second medical device.

18. The method of claim 16, the method further comprising:
determining, by the processing circuitry, a value of a first parameter based on the separated first source signal;
determining, by the processing circuitry, a value of a second parameter based on the separated second source signal; and
determining, by the processing circuitry, the health condition status of the patient based at least in part on one or more of the value of the first parameter or the value of the second parameter.

19. The method of claim 16, wherein the first source signal is an electrocardiogram (ECG) signal of the patient, and
wherein the second source signal is an ECG signal generated by a fetus in the patient.

20. The method of claim 19, the method further comprising determining the health condition status of the fetus in the patient based on the separated second source signal.
